(19) United States
(12) Patent Application Publication
(10) Pub. No.: US 2024/0026291 A1
(43) Pub. Date: Jan. 25, 2024

(54) DEVICE AND METHOD FOR PREPARING A CELL SUSPENSION

(71) Applicant: CUTISS AG, Schlieren (CH)

(72) Inventors: Vincent RONFARD, Villarzel (CH); Joaquin URDINEZ, Zürich (CH); Claude HOLENSTEIN, Zürich (CH)

(73) Assignee: CUTISS AG, Schlieren (CH)

(21) Appl. No.: 18/255,374

(22) PCT Filed: Dec. 1, 2021
(86) PCT No.: PCT/EP2021/083810

§ 371 Date: Jun. 1, 2023

(30) Foreign Application Priority Data
Dec. 1, 2020 (EP) 20211094.6

(51) Int. Cl. C12N 5/071; C12M 1/00; C12N 5/00

(52) U.S. Cl. CPC C12N 5/0626; C12M 45/09; C12M 45/06; C12M 23/40; C12M 29/06; C12N 5/0087; C12N 2509/00

(57) ABSTRACT

The invention relates to a device (1) for preparing a cell suspension comprising an enzyme reservoir (10), a first purification compartment (20) for receiving a first purification material (P1), wherein the first purification compartment (20) is in fluid connection or can be brought into fluid connection with the enzyme reservoir (10), a syringe (40) comprising a barrel (41) defining a barrel compartment (45) and a piston (42) which is movably arranged in the barrel compartment (45), wherein the syringe (40) comprises a syringe inlet (43) connected to the barrel compartment (45), wherein the syringe inlet (43) is in fluid connection or can be brought into fluid connection with the first purification compartment (20), and wherein the syringe (40) comprises a syringe outlet (44), and wherein the device (1) comprises a flow path from the enzyme reservoir (10) via the first purification compartment (20) to the syringe inlet (43). Also provided herein are methods for preparing a cell suspension.

The invention relates to a device and a method for preparing a cell suspension, in particular for treatment of skin conditions, e.g. vitiligo, depigmentation or burn scars, by injection of the prepared cell suspension into an area of the skin affected by the skin condition. In particular, the device is suitable for preparation of a cell suspension from a subject and on-site injection into skin of the same subject (autologous cell transfer), particularly without cultivating the cells of the cell suspension in between.

Some skin conditions can be treated according to methods of the prior art by injecting certain cell types in a specific layer of the skin.

For example, vitiligo is a disease associated with depigmentation of areas of the skin, which is believed to be caused by an autoimmune response against melanin-producing melanocyte cells. It has been shown that vitiligo can be treated by microneedle injection of a melanocyte suspension into the epidermis of the affected areas, which eventually results in repigmentation (Regazzetti C, Alcor D, Chignon-Sicard B, Passeron T, Pigment Cell Melanoma Res. 29, 481-483, 2016; Lagrange S, Montaudié H, Fontas E, Bahadoran P, Lacour J-P, Passeron T: British Journal of Dermatology 180, 1539-1540, 2019).

To treat skin conditions such as, e.g. vitiligo, depigmentation and burn scars, it is highly desirable to obtain the injected cell suspension from the same patient that is to be treated, i.e. to use an autologous cell transfer. In contrast to allogenic transfer, this has the advantages that no immune response occurs, that there is no danger of rejection of the transplanted cells, and that there is no risk of contamination with pathogens (e.g. viruses like HIV).

Some devices and methods to prepare cell suspensions from skin samples for autologous cell transfer have been described in the prior art. For example, a device with a heating means and a filter unit for semi-automatic cell preparation by enzymatic digestion and subsequent filtration as well as a corresponding preparation protocol have been disclosed in EP 1 357 922 B1. A similar further automated device and corresponding method based on mechanical cell disintegration are described in EP 2 970 856 B1.

However, in particular, these devices and methods have the disadvantage that a cell mixture is obtained, which may contain cell types which are not desired for a specific treatment. In addition, the composition of the resulting cell mixture is dependent on the type of skin sample and the preparation protocol and is thus not always known or controllable and not always reproducible, which may lead to varying success of therapy.

Therefore, the objective underlying the present invention is to provide a device and a method for preparing a cell suspension from skin samples for autologous transfer which is improved in view of the above-stated disadvantages of the prior art.

This objective is attained by the subject matter of the independent claims 1 (device) and 12 (method). Embodiments of the invention are stated in sub claims 2 to 11 and 13 to 15 and are described hereafter.

A first aspect of the invention relates to a device for preparing a cell suspension comprising an enzyme reservoir for containing an enzyme solution capable of digesting components of a tissue sample, particularly a skin sample, to obtain a cell suspension. The device further comprises a first purification compartment for receiving a first purification material capable of binding and/or retaining at least one component of the cell suspension, wherein the first purification compartment is in fluid connection or can be brought into fluid connection with the enzyme reservoir. The device further comprises a syringe comprising a barrel defining a barrel compartment and a piston which is movably arranged in the barrel compartment, wherein the syringe comprises a syringe inlet connected to the barrel compartment, and wherein the syringe comprises a syringe outlet for dispensing the cell suspension from the barrel compartment. The device comprises a flow path from the enzyme reservoir via the first purification compartment to the syringe inlet, such that the cell suspension, components of the cell suspension, buffer solutions and the like can flow from the enzyme reservoir via the first purification compartment towards the syringe inlet.

In particular, the cells can be injected into skin areas to be treated through needles or similar means coupled to the syringe outlet.

The barrel compartment of the syringe may be rigidly or flexibly coupled (e.g. by a flexible tubing) to the flow path of the device, particularly to allow better handling and facilitate accessibility of different body sites.

Due to the first purification material, it is possible to obtain a purified cell suspension containing cell types of interest, from which unwanted cells and other material are at least partly, particularly substantially, removed, on site for immediate autologous cell transfer, particularly without the need to cultivate the cells between taking the tissue sample and injecting the cell suspension.

Furthermore, the syringe for injection of the cell suspension is directly coupled to the flow path of the cell preparation device which obviates the need for a transfer step. Additionally, according to some embodiments of the invention, the syringe may be used to generate a vacuum in the flow path from the enzyme reservoir to the syringe inlet to move the cell suspension through the different reservoirs and compartments simply by pulling the piston of the syringe. Alternatively, for example, the device may be set up, such that the cell suspension, components of the cell suspension, buffer solutions and the like flow through the flow path as a result of gravitational force, i.e. the enzyme reservoir may be arranged above the syringe inlet during preparation of the cell suspension.

In certain embodiments, the device comprises at least one valve arranged in a flow path between the syringe inlet and the enzyme reservoir.

In certain embodiments, the device comprises a filter arranged in the flow path downstream of the enzyme reservoir, wherein the filter is configured to retain cell aggregates, particularly wherein the filter is configured to only allow single cells to access the first purification compartment.

In certain embodiments, the at least one valve is a switchable valve which is configured to be switched between an open state allowing flow through the valve and a closed state blocking flow through the valve. Such a switchable valve may be actuated manually or automatically to switch between the open and the closed state. By a switchable valve, it is possible to selectively open and close different parts of the flow path at a desired time during preparation of the cell suspension.

In certain embodiments, the at least one valve is configured to allow flow in a first flow direction from the enzyme reservoir through the first purification compartment to the barrel compartment, and the at least one valve is configured to block flow in a second flow direction opposite the first flow direction. In other words, the at least one valve is a check valve according to this embodiment. This feature advantageously prevents an unwanted backflow of the cell suspension, and therefore improves the performance of the device.

In certain embodiments, the first purification material is an affinity matrix capable of selectively binding at least one cell type, particularly keratinocytes or melanocytes.

In certain embodiments, the first purification material comprises affinity molecules, particularly antibodies, which selectively bind to keratinocytes or melanocytes.

In certain embodiments, the first purification material comprises affinity molecules, particularly antibodies, which bind keratinocytes but do not bind melanocytes or which bind melanocytes but do not bind keratinocytes.

In certain embodiments, the first purification material comprises affinity molecules, particularly antibodies, which selectively bind mesenchymal stem cells or hair follicle cells.

Such an affinity matrix allows to select an affinity molecule binding to a specific cell type which is to be removed from the cell suspension or separated from unwanted components and subsequently eluted from the affinity molecules to generate a purified cell suspension, thereby improving the quality of the final cell suspension which is to be injected into the target site.

In certain embodiments, the first purification material comprises an inhibitor capable of inhibiting an enzymatic activity, particularly an enzymatic activity of an enzyme comprised in the enzyme solution provided in the enzyme reservoir of the device.

This prevents damage to the cells of the cell suspension through unwanted enzymatic digestion e.g. of surface proteins, thus improving the quality of the cell suspension.

In certain embodiments, the device comprises a second purification compartment for receiving a second purification material capable of binding and/or retaining at least one component of the cell suspension, particularly wherein the second purification compartment is arranged in the flow path between the first purification compartment and the syringe inlet.

The second purification compartment makes the preparation method more versatile since it allows an additional purification step. In particular, by the second purification component, it is possible to either remove a second unwanted cell type (e.g. in case of an affinity matrix) or remove unwanted small molecules (e.g. in case of a size exclusion material) from the suspension, which might be problematic if injected into the target skin site. Thereby, the quality of the suspension is further improved.

In certain embodiments, the second purification material is a size exclusion material capable of separating components of the cell suspension according to their molecular weight and/or hydrodynamic radius.

In certain embodiments, the device comprises a buffer reservoir for storing a buffer, wherein the buffer reservoir is in fluid connection or can be brought in fluid connection with the flow path between the enzyme reservoir and the syringe inlet.

In certain embodiments, the device comprises a three-way buffer valve connecting the buffer reservoir to a flow path between the first purification compartment and the second purification compartment.

In certain embodiments, the device comprises a waste reservoir which is in fluid connection or can be brought in fluid connection with the flow path between the enzyme reservoir and the syringe inlet. In particular, the waste reservoir is in fluid connection or can be brought into fluid connection with the flow path between the second purification compartment and the barrel compartment.

In certain embodiments, the device comprises a waste valve, particularly a three-way waste valve, connecting the waste reservoir to the flow path between the enzyme reservoir and the syringe inlet.

In certain embodiments, the device comprises at least one magnet arranged adjacent to the flow path between the enzyme reservoir and the syringe inlet, wherein the at least one magnet is configured to bind magnetic particles comprised in the first purification material and/or the second purification material. In particular, the at least one magnet is arranged in the first purification compartment or the second purification compartment. In particular, the at least one magnet is arranged adjacent to a conduit forming at least a part of the flow path between the enzyme reservoir and the syringe inlet, wherein the at least one magnet and the conduit are arranged with respect to each other, such that a magnetic field is provided in the conduit by the at least one magnet.

By means of the magnetic field provided by the at least one magnet, a species of interest bound to the magnetic particles of the first purification material can be retained and particularly enriched near the at least one magnet. Subsequently, the magnetic particles can be released by removing the species of interest from the magnetic particles (e.g. by changing the ionic strength or the pH in case of antibody-coupled magnetic particles to release the species of interest from affinity molecules, particularly antibodies, coupled to the magnetic particles) or by changing the magnetic field of the at least one magnet, such that the magnetic particles with the bound species are eluted. Alternatively, the magnetic particles (and particularly the affinity molecules coupled to the particles) may be selected such that unwanted components of the initial cell suspension, e.g. cells which are to be removed from the cell suspension, bind to the magnetic particles. In this case, the flow through passing along the magnet containing cells of interest which do not bind to the magnetic particles can be used to generate the final cell suspension, whereas the fraction bound to the magnetic particles can e.g. be discarded.

In certain embodiments, the at least one magnet comprises a switchable electromagnet or a permanent magnet configured to be moved with respect to the conduit. Both mechanisms can be used to change the intensity or switch off the magnetic field and release the magnetic particles from the at least one magnet.

In certain embodiments, the device comprises a mixing chamber arranged in the flow path between the enzyme reservoir and the syringe inlet, and a mixing device configured to mix the cell suspension with the first purification material and/or the second purification material in the mixing chamber.

In certain embodiments, the mixing chamber is arranged in the flow path between the enzyme reservoir and the first purification compartment. In certain embodiments, the mixing chamber is arranged in the flow path between the first purification compartment and the second purification compartment. In certain embodiments, the mixing chamber is arranged in the flow path between the first purification compartment and the syringe inlet. In certain embodiments, the mixing chamber is arranged in the flow path between the second purification compartment and the syringe inlet.

In certain embodiments, the mixing device comprises at least one mixing blade connected to a rod extending along a first longitudinal axis along which the device, particularly a main body of the device, extends, wherein the at least one mixing blade extends from the rod in a radial direction in respect of the first longitudinal axis, such that the at least one mixing blade is rotatable in a circumferential direction in respect of the rod when the rod is rotated around the longitudinal axis. In particular, an end of the rod is connected to a rotary actuator arranged at the outside of the device, wherein rotary actuator is configured to rotate the rod around the first longitudinal axis when the rotary actuator is manually rotated around the first longitudinal axis. Alternatively, in particular, the rod is rotatable by a motor, e.g. by a coupling at an end of the rotor which is connectable to a motor shaft.

In certain embodiments, the device extends along the first longitudinal axis, wherein the device comprises a plurality of layers arranged along the first longitudinal axis, wherein neighboring layers are separated by a vertical divider, wherein each layer forms at least one compartment, wherein particularly the enzyme reservoir, the first purification compartment, the second purification compartment, the at least one further purification compartment, the mixing chamber, the buffer reservoir and/or the waste reservoir are formed by one of said compartments.

In certain embodiments, the device further comprises at least one horizontal divider separating a compartment formed within one of said layers into at least two sub-compartments perpendicular to the first longitudinal axis, wherein particularly the enzyme reservoir, the first purification compartment, the second purification compartment, the at least one further purification compartment, the mixing chamber, the buffer reservoir and/or the waste reservoir are formed by one of said sub-compartments.

In certain embodiments, the device comprises a surgical tool for obtaining a tissue sample, particularly a skin sample, wherein the surgical tool is arranged adjacent to an opening of the enzyme reservoir wherein the opening connects the enzyme reservoir to the outside of the device, particularly such that the tissue sample obtained by the surgical tool is automatically provided in the enzyme reservoir.

The attached surgical tool has the advantage that a standalone device is realized, which allows to perform all steps of the autologous cell transfer with a single apparatus without any additional components.

In certain embodiments, the device comprises a pump inlet arranged in the enzyme reservoir, wherein the pump inlet is configured to be connected to a vacuum pump to generate a vacuum in the enzyme reservoir, such that a portion of skin of a subject is sucked into the enzyme reservoir by the vacuum if the portion of skin is placed on the opening of the enzyme reservoir, and wherein the surgical tool comprises at least one movable, particularly rotatable, blade assembly configured to cut off the portion of skin, thereby obtaining the tissue sample. In particular, the device is configured such that the tissue sample can be provided in the enzyme reservoir by the vacuum. For example, the vacuum could be induced by an existing pressured air outlet provided in a hospital.

In certain embodiments, the device comprises an opening for inserting a tissue sample into the enzyme reservoir, particularly wherein the device comprises a lid for closing the opening.

In certain embodiments, the device comprises at least one sterile filter, particularly having a pore size of 0.22 μm or less, wherein the sterile filter is arranged at an opening to the exterior, particularly an opening connecting the flow path to the exterior, wherein particularly the filter covers the opening.

In certain embodiments, the device comprises an injector comprising a plurality of hollow needles, particularly microneedles, wherein the injector is connected to the syringe outlet, such that the cell suspension is dispensable from the barrel compartment through the needles.

In certain embodiments, each of the needles has a length of 100 μm to 500 μm, particularly 100 μm to 200 μm.

In certain embodiments, the device is sterile (i.e., sterilized) and/or disposable.

In certain embodiments, the device comprises at least a first part and a second part, wherein the first part is disposable and the second part is non-disposable.

A second aspect of the invention relates to a method for preparing a cell suspension comprising the steps of providing a tissue sample, particularly a skin sample, in the enzyme reservoir of the device according to the first aspect, providing an enzyme solution in the enzyme reservoir, wherein the enzyme solution is capable of digesting components of the tissue sample, incubating the tissue sample in the enzyme solution to digest components of the tissue sample, thereby obtaining a cell suspension, providing the first purification material in the first purification compartment, and passing the cell suspension from the enzyme reservoir through the first purification compartment into the barrel compartment, such that at least one component of the cell suspension is bound or retained by the first purification material, and dispensing the cell suspension from the syringe outlet.

In certain embodiments, the first purification material may comprise a filter material or the device may comprise a filter comprising the filter material, particularly wherein the filter material is capable of retaining cell aggregates, more particularly such that only single cells are obtained.

In certain embodiments, the tissue sample is a biopsy, particularly a full thickness biopsy or a split thickness biopsy. In certain embodiments, the tissue sample comprises hair follicles or hair, particularly plucked from a part of the human body. In certain embodiments, the tissue sample is an epidermal blister.

In certain embodiments, at least one enzyme comprised in the enzyme solution is active at a temperature of 20-25° C. For example, engineered variants of trypsin with these properties have been described and are commercially available. Using such enzymes has the advantage that the entire autologous cell transfer and preparation procedure can be performed at room temperature without additional heating means.

In certain embodiments, the cell suspension is passed from the enzyme reservoir through the first purification compartment into the barrel compartment by moving the piston in the barrel compartment to reduce the pressure in the barrel compartment.

In certain embodiments, the cell suspension is passed from the enzyme reservoir through the first purification compartment into the barrel compartment by gravitational force.

In certain embodiments, the cell suspension is dispensed from the syringe outlet by moving the piston in the barrel compartment to increase the pressure in the barrel compartment.

In certain embodiments, the first purification material and/or the second purification material is or comprises an affinity material capable of selectively binding at least one cell type, particularly keratinocytes or melanocytes, wherein more particularly the affinity material comprises antibodies which selectively bind to keratinocytes or melanocytes, wherein particularly the first purification material comprises an inhibitor capable of inhibiting an enzymatic activity, particularly an enzymatic activity of an enzyme comprised in the enzyme solution.

In certain embodiments, the first purification material is an affinity material capable of selectively binding melanocytes, wherein the first purification material does not bind to keratinocytes.

In certain embodiments, the first purification material is an affinity material capable of selectively binding keratinocytes, wherein the first purification material does not bind to melanocytes.

In certain embodiments, after passing the cell suspension through the first purification compartment and before dispensing the cell suspension from the syringe outlet, a second purification material is provided in the second purification compartment, and the cell suspension is passed through the second purification compartment into the barrel compartment, such that at least one component of the cell suspension is bound or retained by the second purification material.

In certain embodiments, the cell suspension is passed from the first purification compartment through the second purification compartment into the barrel compartment by moving the piston in the barrel compartment to reduce the pressure in the barrel compartment. Alternatively, the cell suspension flows from the first purification compartment through the second purification compartment into the barrel compartment driven by gravitational force.

In certain embodiments, the second purification material is a size exclusion material capable of separating components of the cell suspension according to their molecular weight and/or hydrodynamic radius.

In certain embodiments, the first purification material and/or the second purification material comprises magnetic particles, particularly magnetic particles coupled to an affinity material capable of selectively binding a species of interest in the cell suspension.

In certain embodiments, the first purification material and the cell suspension are mixed in the mixing chamber, thereby obtaining a mixture, wherein the mixture is provided in the first purification compartment or in the second purification compartment, and wherein at least one magnet is provided in the first purification compartment or the second purification compartment, such that a species of interest bound to the magnetic particles, particularly bound to the affinity molecule coupled to the magnetic particles, is retained in the first or second purification compartment by a magnetic field provided by the at least one magnet. In particular, a washing buffer is provided in the first or second purification compartment to wash the magnetic particles after retaining the magnetic particles by the at least one magnet. In particular, the bound species is removed from the magnetic particles, particularly by providing an elution buffer capable of weakening the interaction between the magnetic particles and the bound species (e.g. by low pH or high ionic strength) or by reducing the magnetic field acting on the magnetic particles by the at least one magnet (e.g., by moving a permanent magnet or by controlling the voltage through an electromagnet).

In particular, the first purification material comprising the magnetic particles is stored in a storage compartment, wherein the first purification material or the second purification material is provided in the first purification compartment, the second purification compartment or the mixing chamber from the storage compartment to mix the first purification material or the second purification material with the cell suspension.

In certain embodiments, the dispensed cell suspension comprises melanocytes. In certain embodiments, the dispensed cell suspension is enriched in melanocytes or is a pure suspension of melanocytes.

In certain embodiments, the dispensed cell suspension comprises mesenchymal stem cells or hair follicle cells.

In certain embodiments, the dispensed cell suspension comprises a reduced amount of keratinocytes, wherein particularly the dispensed cell solution is substantially or completely free from keratinocytes.

In certain embodiments, the cell suspension is injected into a target site of the skin of a subject through at least one needle connected to the syringe outlet upon dispensing the cell suspension from the syringe outlet.

In certain embodiments, the cell suspension is injected into a target site of the skin of a subject by the injector comprising the plurality of hollow needles, particularly microneedles, connected to the syringe outlet upon dispensing the cell suspension from the syringe outlet.

Wherever alternatives for single separable features are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

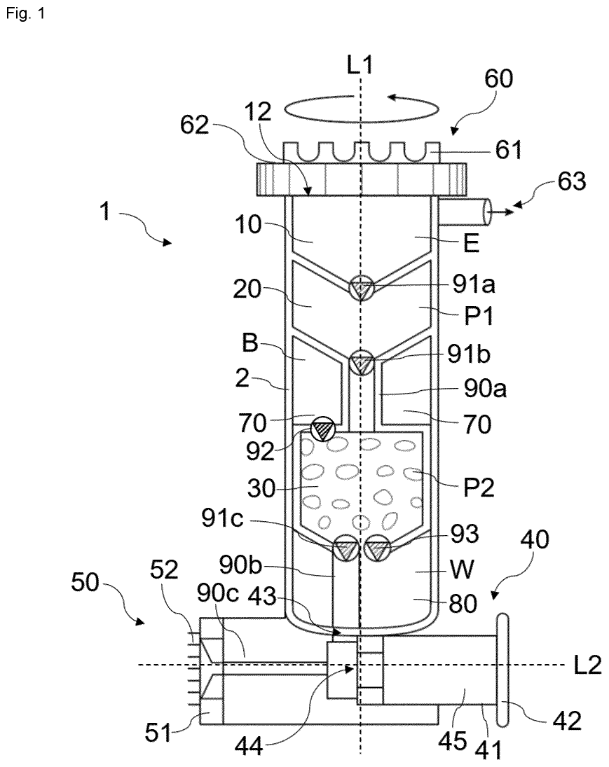

FIG. 1 shows a schematic of a device for preparing a cell suspension according to a first embodiment of the invention;

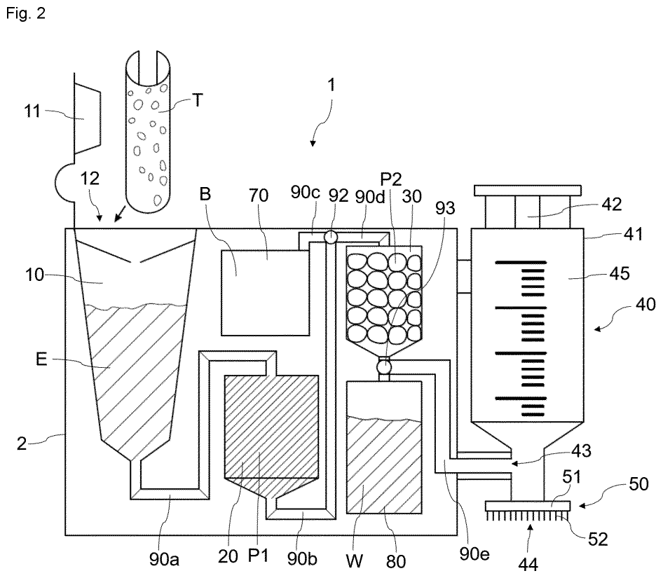

FIG. 2 shows a schematic of a device for preparing a cell suspension according to a second embodiment of the invention;

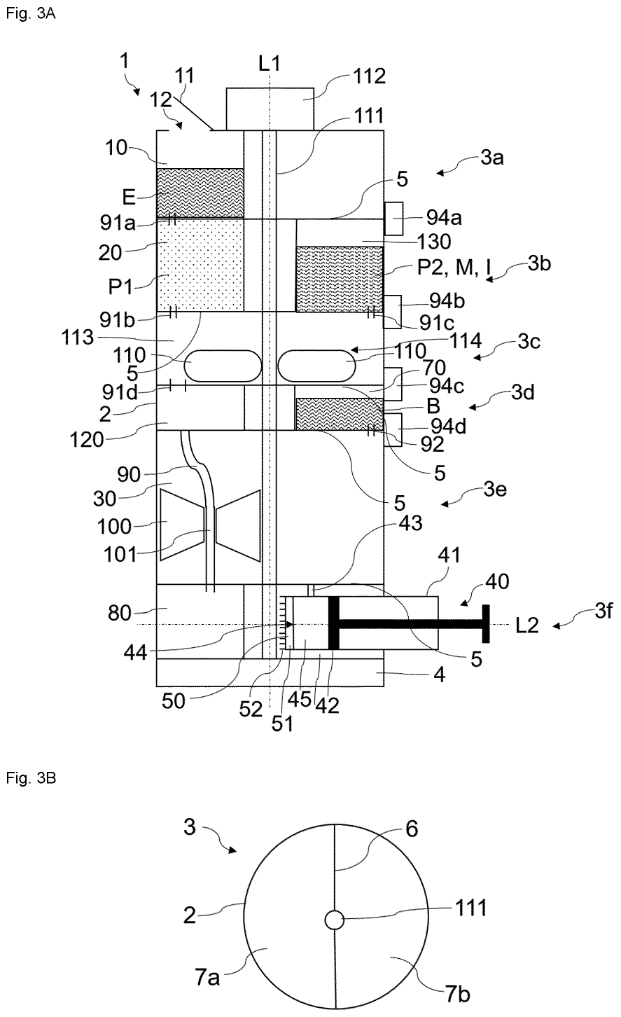

FIG. 3 shows a schematic of a device for preparing a cell suspension according to a third embodiment of the invention;

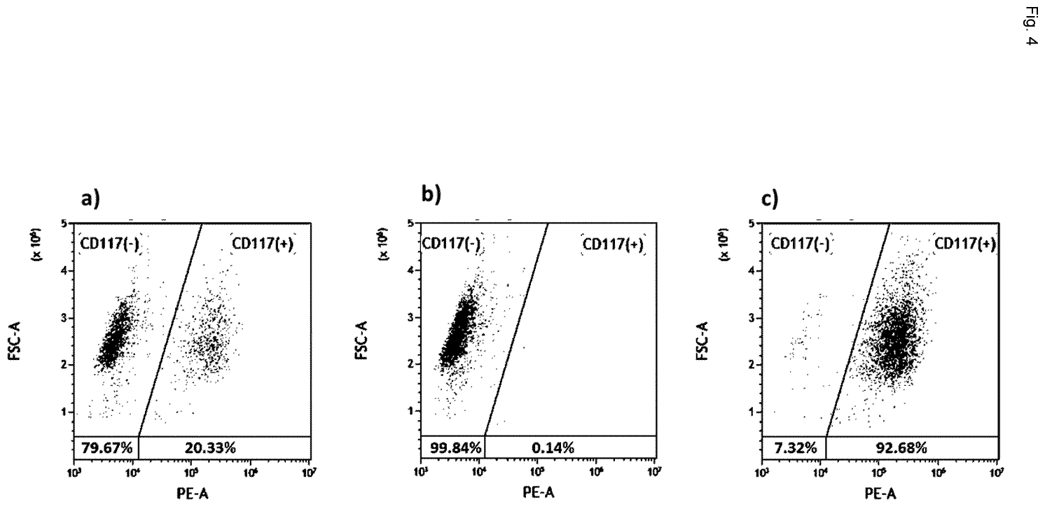

FIG. 4 depicts representative flow cytometry images showing the presence of keratinocytes (CD117(−)) and melanocytes (CD117(+)) in the cell mixture before being loaded to the column (FIG. 4A); the flow-through containing non-bound cells (FIG. 4B), and in the eluted cells that were magnetically retained by the column (FIG. 4C). The numbers indicate the relative abundance of each cell type to the total number of cells analyzed.

FIG. 1 shows a schematic sectional view of a device 1 for preparing a cell suspension comprising a main body 2 extending along a first longitudinal axis L1. The device 1 comprises an enzyme reservoir 10, a first purification compartment 20 and a second purification compartment 30 formed inside the main body 2 and arranged along the first longitudinal axis L1 from top to bottom in FIG. 1. A surgical tool 60 is attached to the main body 2.

The device 1 further comprises a syringe 40 arranged on a bottom end of the main body 2, the syringe extending along a second longitudinal axis L2 which is perpendicular to the first longitudinal axis L1. A syringe inlet 43 of the syringe 40 is in fluid connection with the second purification compartment 30. The syringe 40 further comprises a syringe outlet 44 connected to an injector 50, particularly comprising a plurality of needles 52 (such as microneedles) attached to a support 51.

The surgical tool 60 comprises a rotatable blade assembly 61 comprising a plurality of blades arranged in a circumferential direction with respect to the first longitudinal axis L1. The rotatable blade assembly 61 is arranged on and fixed to a solid base 62 which is connected to the main body 2. The rotatable blade assembly 61 is arranged around an opening 12 leading to the enzyme reservoir 10 of the device 1. A pump inlet 63 for connecting a vacuum pump branches off from the wall of the main body 2 forming the enzyme reservoir 10.

To prepare a tissue sample T, the surgical tool 60 can be placed on the skin surface of a subject, a vacuum pump can be connected to the pump inlet 63 and a vacuum can be generated by the vacuum pump in the enzyme reservoir 10, such that a portion of skin of the subject is partially sucked into the enzyme reservoir 10 of the device 1. By rotating the rotatable blade assembly 61 around the longitudinal axis L1, for example manually or driven by a motor, the portion of skin can be cut, thereby generating a tissue sample T in form of a micro blister, which is then automatically sucked into the enzyme reservoir 10 by means of the vacuum.

Before or after providing the tissue sample T in the enzyme reservoir 10, an enzyme solution E is provided in the enzyme reservoir 10 to digest components of the tissue sample T, for example components of the extracellular matrix, to obtain a cell suspension, particularly containing keratinocytes and melanocytes. The enzyme solution E may contain a single enzyme or a mixture of enzymes, e.g. trypsin or a trypsin derivative. To obtain digestion of the matrix components, the device 1 is particularly incubated at a temperature, where the enzyme or enzymes in the enzyme solution E is or are active (e.g., room temperature or 37° C.), and in particular for a time, which is sufficient to completely degrade components of the extracellular matrix and obtain a cell suspension devoid of these unwanted components.

Hereafter, the obtained cell suspension is moved from the enzyme reservoir 10 to the first purification compartment 20.

To this end, for example, a vacuum may be provided at the downstream end of the device, e.g. by pulling the piston 42 of the syringe 40 while the syringe inlet 43 connected to the second purification compartment 30 is open and the syringe outlet 44 towards the injector 50 is closed.

Alternatively, the cell suspension may be moved to the first purification compartment 20 by a vacuum generated by a vacuum pump or by applying pressure at the upstream end, i.e. the opening 12 or the pump inlet 63.

Yet alternatively, the device may be set up such that the cell suspension flows into the first purification compartment 20 driven by gravitational force, i.e. in case the enzyme reservoir 10 is arranged above the first purification compartment 20.

A valve 91a, e.g., a check valve or a switchable valve, may be provided upstream of the first purification compartment 20 to allow flow in the direction from the enzyme reservoir 10 to the first purification compartment 20, but block flow in the opposite direction (in case of a check valve) or to allow flow from the enzyme reservoir 10 to the first purification compartment 20 when switched to an open state but block flow in a closed state.

The first purification compartment 20 contains a first purification material P1, e.g. an affinity matrix which specifically binds to certain cell types in the cell suspension, such as keratinocytes or melanocytes. To this end, the first purification material P1 may be provided in the form of beads, to which antibodies specific to certain cell types, e.g. keratinocytes or melanocytes, are coupled. In addition, the first purification material P1 may contain an inhibitor capable of inhibiting at least one enzyme contained in the enzyme solution E to ensure that digestion by the enzyme solution E does not continue to an unwanted extent. The inhibitor may be immobilized on the first purification material P1, e.g., the antibody coupled beads, or may be provided in soluble form.

In particular, the aim of the first purification material P1 is to remove a certain cell type, such as keratinocytes, from the cell suspension to purify the cell suspension. For example, if the tissue sample T mainly contains keratinocytes and melanocytes, the aim of the first purification material P1 may be to remove the keratinocytes from the cell suspension completely, but keep the melanocytes in the suspension flowing through the first purification material P1.

Alternatively, the first purification material P1 may bind to a certain first cell type of interest, such as melanocytes, but may not bind to a second cell type, such as keratinocytes, which should be removed from the final cell suspension. In this case, the flowthrough of the first purification material P1 may be discarded, and the bound cells of interest may be eluted from the first purification material P1 in a subsequent step, e.g. by applying a suitable elution buffer to the first purification material P1.

In particular, both procedures may be used to obtain a pure suspension of melanocytes or a suspension which is enriched in melanocytes.

According to an optional further step of the method according to the invention, the partially purified suspension is moved via a further valve 91b and a conduit 90a to the second purification compartment 30 containing a second purification material P2.

Similar to moving the cell suspension to the first purification compartment 20 described above, this may be achieved by providing a vacuum downstream of the second purification compartment 30, e.g. by pulling the piston 42 of the syringe 40, by providing pressure upstream of the second purification compartment 30 or by gravity flow.

For example, the second purification material P2 may be a size exclusion material, e.g. formed by spherical particles having a defined diameter and pore size. Such a size exclusion material is capable of separating a solution or suspension according to molecular size and/or hydrodynamic radius, typically, such that small molecules, which are able to enter pores in the spherical particles are retained, whereas larger species such as cells flow around the size exclusion material, and are therefore eluted first. This principle may be utilized in the second purification compartment 30 to separate the pre-purified cell suspension from unwanted small molecules, such as remaining active or inactive enzymes or salts which must be removed before injection of the cell suspension into the human or animal body.

Further optionally, the second purification compartment 30 may be connected to a waste reservoir 80 for receiving a waste solution W, i.e. by a waste valve 93, particularly a switchable valve. In particular, by opening the waste valve 93 and flowing a buffer solution through the second purification compartment 30 (and the second purification material P2 arranged therein), unwanted components of the cell suspension, i.e. small molecules, may be removed from the second purification material P2, wherein the flowthrough is retained in the waste reservoir 80. For example, the buffer solution may be stored in a buffer reservoir 70 which can be connected to the second purification compartment 30, e.g. by a buffer valve 92.

On its downstream end, the second purification compartment 30 is further connected to the syringe inlet 43 leading to the barrel compartment 45 of the syringe 40 via a further valve 91c and a conduit 90b.

Optionally, in case the valves 93 and 91c are switchable valves, unwanted components of the cell suspension can be first removed from the second purification material 30 by opening valve 93 to the waste reservoir 80 and the final cell suspension can be subsequently moved into the barrel compartment 45 of the syringe by opening valve 91c.

The injector 50 at the end of the syringe 40 may then be placed on a skin area of the subject to be treated (in particular the same subject from which the tissue sample T was obtained, in other words to achieve an autologous cell transfer), such that the needles 52 (which are particularly formed as microneedles) pierce the stratum corneum, and the tips of the needles 52 extend to the skin layer of interest, such as the epidermis or dermis depending on the needle length. The piston 42 of the syringe 40 is then moved to inject the cell suspension from the barrel compartment 45 through the syringe outlet 44, the conduit 90c and the injector 50 into the respective layer of the skin, e.g. the epidermis (i.e. using a needle length of about 100 μm to 200 μm).

In case of a melanocyte cell suspension, this procedure can be applied to treat areas of the skin affected by vitiligo or depigmentation. However, other cell types can be prepared to treat other diseases or disorders, particularly skin diseases or disorders.

FIG. 2 is a schematic view of a further embodiment of the device 1 for preparing a cell suspension. Similar to the device 1 shown in FIG. 1, this embodiment comprises a main body 2 comprising or encasing an enzyme reservoir 10 filled with an enzyme solution E, a first purification compartment 20 containing a first purification material P1, a second purification compartment 30 containing a second purification material P2, a buffer reservoir 70 and a waste reservoir 80.

In the embodiment according to FIG. 2, the enzyme reservoir 10, the first purification compartment 20 and the second purification compartment 30 are connected via conduits 90a, 90c, 90d, 90e, which may be formed e.g. by channels within a solid main body 2 or by tubing encased by the main body 2.

Furthermore, the device 1 comprises a syringe 40 with a barrel 41 forming a barrel compartment 45 and a piston 42 which is movably arranged in the barrel compartment 45, wherein the syringe 40 comprises a syringe inlet 43 leading to the barrel compartment 45, wherein the syringe inlet 43 is in fluid connection with the downstream side of the second purification compartment 30. The syringe 40 further comprises a syringe outlet 44 connected to an injector 50 comprising a support 51, to which an array of needles 52, e.g., microneedles, is connected.

In contrast to the embodiment shown in FIG. 1, the device shown in FIG. 2 does not contain a surgical tool 60, but instead, the enzyme reservoir 10 is accessible from the outside of the device 1 via an opening 12, which is closable by a lid 11.

The tissue sample T, which may be obtained e.g. by a separate surgical tool, a scalpel, a biopsy needle, a dermatome or the like, can be provided in the enzyme reservoir 10 from the outside through this opening 12 and is then digested by the enzyme solution E as described above. Furthermore, the cell suspension obtained by the digestion of the tissue sample T may be moved through the different reservoirs and compartments of the device 1 either by applying a vacuum on the downstream side, e.g. by pulling the piston 42 of the syringe 40, by applying pressure on the upstream side, e.g., via the opening 12 of the enzyme reservoir 10, or by gravity flow, as described above.

The device 1 shown in FIG. 2 comprises a buffer reservoir 70 for containing a buffer solution, wherein the buffer reservoir 70 is connected to a flow path between the first purification compartment 20 and the second purification compartment 30 by a three-way buffer valve 92.

By opening the three-way buffer valve 92 in the direction between the buffer reservoir 70 and the second purification compartment 30 and by further opening a three-way waste valve 93 arranged between the second purification compartment 30, the waste reservoir 80 and the barrel compartment 45, unwanted components of the cell suspension, e.g. small molecules or protein components used for enzyme neutralization, may be washed off the second purification material P2 into the waste reservoir 80 to be removed from the cell suspension. This has the advantage that these components do not end up in the cell suspension that is injected by the injector 50, thereby avoiding potential health problems.

Additionally or alternatively, buffer solution can be applied to the second purification material P2 from the buffer reservoir 70 while the three-way waste valve 93 is open between the second purification compartment 30 and the barrel compartment 45 to elute (e.g., push out) the cell suspension from the second purification material P2, particularly after gravitational separation, and move the cell suspension into the barrel compartment 45 of the syringe 40 for subsequent injection.

In case the second purification material P2 is a size exclusion material, the buffer from the buffer reservoir 70 may be applied to dilute the cell suspension prior to being loaded on the size exclusion material in the second purification compartment 30 to avoid potential clogging of the size exclusion matrix. This dilution may be achieved by mixing the cell suspension and the buffer solution downstream of the three-way buffer valve 92 and upstream of the second purification compartment 30.

Purification of the cell suspension and injection into a skin area to be treated may be performed with the device 1 according to the second embodiment shown in FIG. 2 in an identical or similar manner to that described above for the embodiment shown in FIG. 1

FIG. 3 illustrates a further embodiment of the device 1, wherein FIG. 3a shows a section of the device 1 parallel to the first longitudinal axis L1 and FIG. 3B shows an exemplary layer 3 of the device 1 in a sectional view perpendicular to the first longitudinal axis L1.

According to the embodiment shown in FIG. 3, the device 1 comprises a main body 2 which is composed of a plurality of layers 3a, 3b, 3c, 3d, 3e, 3f arranged above a base 4 along the first longitudinal axis L1.

The layers 3a, 3b, 3c, 3d, 3e, 3f are separated by vertical separators 5 (integrally formed with the main body 2 or provided as separate components), wherein the vertical separators 5 extend perpendicular to the first longitudinal axis L1. Due to the vertical separators 5, separate compartments are provided by the layers, wherein certain compartments are connected by valves 91a, 91b, 91c, 91d, 92, 93 arranged in openings of the vertical separators 5.

As schematically shown for layer 3 in FIG. 3B, the layers 3a, 3b, 3c, 3d, 3e, 3f may comprise additional horizontal separators 6 separating the compartment formed by a layer into a plurality of sub-compartments 7a, 7b. Of course, the arrangement shown in FIG. 3B is only an example, and any number of horizontal separators 6 may be used in the embodiment according to FIG. 3, resulting in sub-compartments of any desired shape and size.

The layer 3a of the device 1, particularly one of a plurality of sub-compartments of layer 3a formed by horizontal dividers 7 (not shown), forms an enzyme reservoir 10 for receiving an enzyme solution E. The enzyme reservoir 10 comprises an opening 12 towards the outside of the device 1 which is closable by a lid 11. Similar to the embodiment shown in FIG. 2, a tissue sample T may be obtained e.g. by a separate surgical tool, a scalpel, a biopsy needle, a dermatome or the like, and provided in the enzyme reservoir 10 from the outside through the opening 12. The tissue sample T is then digested by the enzyme solution E as described above.

After completing the enzymatic digest, the valve 91a between the enzyme reservoir 10 and the first purification compartment 20 (which may be a switchable valve) is opened, particularly using actuator 94a, such that the obtained cell suspension flows into the first purification compartment 20 arranged in the layer 3b, e.g. by gravity flow.

In particular, the first purification compartment 20 is formed as a sub-compartment of the layer 3b by corresponding horizontal dividers 7. The first purification compartment 20 contains the first purification material P1, e.g. a filter material which particularly removes cellular debris and extracellular matrix components from the cell suspension if present.

Subsequently, the valve 91b may be opened, such that the filtered cell suspension enters a mixing chamber 113 formed by layer 3c of the device 1, e.g. by gravity flow. A mixing device 114 comprising mixing blades 110 connected to a rotatable rod 111 is arranged in the mixing chamber 113. The rod 111 is arranged parallel to the first longitudinal axis L1 and extends from a rotary actuator 112 through central through-holes of the layers 3a, 3b, 3c, 3d, 3e, 3f towards the base 4. When the rod 111 is rotated by means of the rotary actuator 112 (e.g. manually or by connecting a motor), the blades 110 rotate around the first longitudinal axis L1 in the mixing chamber 113.

A further sub-compartment of the layer 3b above the layer 3c forms a storage compartment 130 containing a second purification material P2, particularly comprising magnetic particles M and optionally an inhibitor solution I for inhibiting enzymes of the enzyme solution E. The magnetic particles M may be coupled to an affinity molecule, such as an antibody, which specifically binds to cells of interest to be obtained by the method of the invention.

The second purification material P2 comprising the magnetic particles M may be provided in the mixing chamber 113, particularly by opening valve 91c between the storage compartment 130 and the mixing chamber 113. Opening of the valve 91c and/or the valve 91b may be performed by actuating actuator 94b.

After adding the second purification material P2 to the cell suspension in the mixing chamber 113, the mixing blades 110 of the mixing device 114 are rotated by means of the rotary actuator 112 coupled to the rod 111 around the first longitudinal axis L1 to mix the cell suspension with the second purification material P2, such that the magnetic particles M bind to certain cells in the cell suspension, particularly by the affinity molecules, e.g. antibodies, coupled to the magnetic particles M.

The mixture of the cell suspension and the second purification material P2 is then collected in a collection chamber 120 formed by layer 3d, particularly a sub-compartment thereof formed by horizontal dividers 7. Optionally the collection chamber 120 is connected to the mixing chamber 113 by valve 91d which may be actuated by actuator 94d.

Layer 3e of the device 1 forms a second purification compartment 30 in which at least one magnet 100 for binding and retaining the magnetic particles M is arranged. Particularly, the second purification compartment 30 further comprises a conduit 90 providing a flow path between the collection chamber 120 and a waste reservoir 80 along the magnet 100, wherein more particularly the conduit extends through a through-hole 101 in the magnet 100 or gap 101 between the magnets 100 (in case of more than one magnet) to achieve close proximity of the cell suspension to the magnet or magnets 100. This results in an increased magnetic field and an improved retention of the magnetic particles M with bound cells of interest.

The conduit 90 may optionally contain a matrix material configured to amplify the magnetic field of the magnet 100 in the conduit, particularly containing porous ferromagnetic particles.

In particular, components of the cell suspension which are not bound to the magnetic particles M flow through the conduit 90 into the waste reservoir 80.

A further sub-compartment of the layer 3d may contain a buffer reservoir 70 which may be coupled to the conduit 90 via the buffer valve 92 (e.g., actuated by actuator 94d). The other end of the conduit 90 may be connected to the syringe inlet 43, such that a flow path between the buffer reservoir 70 and the barrel compartment 45 of the syringe 40 is established.

Buffer may be applied through the conduit 90 to elute the magnetic particles with the bound cells of interest and provide the final cell suspension in the barrel compartment 45. For example, this buffer may be configured to weaken or abolish binding between the affinity molecules coupled to the magnetic particles and the cells of interest, e.g. by an increased ionic strength or a decreased pH compared to the buffer conditions of the second purification material P2.

Alternatively or additionally, the magnetic field in the second purification compartment 30, particularly in the conduit 90, may be weakened or abolished, e.g. by moving the magnet 100 in the second purification compartment 30 (e.g., in case of a permanent magnet) or by switching off the magnet 100 (in case of an electromagnet).

As an alternative to the above-described method, instead of retaining cells of interest by the magnetic particles and removing unwanted cells in the flowthrough, unwanted cells could be retained by the magnetic particles and the flow through passing the magnet 100 could be directly provided in the barrel compartment 45 of the syringe as the final cell suspension.

In particular, after providing the cell suspension in the barrel compartment 45, the syringe 40 is removed from the device 1 and the cell suspension is injected into skin by the injector 50.

### Example 1—Separation of Keratinocytes and Melanocytes Using Magnetic Particles

### Cells and Cell Culture Conditions

Primary human keratinocytes (KC) were isolated as described elsewhere. Briefly, a split-thickness skin biopsy (purchased to ProviSkin) was cut in pieces of approximately 1 cm 2, followed by Dispase digestion (Corning, 3542359) for 1.5 hours at 37° C. Thereafter, the epidermis and the dermis were mechanically separated. Epidermal pieces were digested with (Sigma-Aldrich, 59418C-100) for 2 min at 37° C. and filtered through a 100 μm cell strainer. Keratinocytes were cultivated in CellNTec medium (CnT-BM.1 supplemented with CnT-07HC.S, CnT-07HC.S, CnT-07HC.S, Amphotericin B and Gentamicin) in 5% CO₂/95% air atmosphere with saturated humidity at 37° C. until reaching passage n3. The purity of the keratinocytes was determined as >99.9% assessed by the keratinocyte-specific marker PanK5/K8 (data not shown).

Human melanocytes (MC) where purchased from Promocell (C-12400) and expanded in Melanocyte Growth media (Promocell, C-24010) as recommended by the manufacturer, until passage n6-9. Both cell types, keratinocytes and melanocytes, were frozen in Cell Freezing Medium-DMSO Serum free (Sigma-Aldrich, C6295) and stored in liquid nitrogen for 3-5 weeks until the day of the experiment. On that day, cells were quickly thawed at 37° C. and washed once with Dulbecco's Modified Eagle medium (DMEM) supplemented with 10% fetal bovine serum (FBS), followed by a centrifugation round and a media replacement. Resuspended cells where passed through a 100 μm cell strainer and its concentration and viability were assessed in duplicates. Cells were mixed to have a final cell suspension of 5×10⁶ live cells, being 80% of them keratinocytes, and the remaining 20% melanocytes (4:1).

### Magnetic Cell Separation

### Column Preparation

The magnetic separation of cells was done with an LD Column (Miltenyi, 130-042-901) following manufacturer's instructions. Briefly, the column was placed on a magnetic stand (MidiMACS Separator—130-042-302, Miltenyi) and washed once with Column Buffer (PBS, pH 7.2, 0.5% bovine serum albumin, 2 mM EDTA). 4×10⁶ cells (keratinocyte and melanocyte mixture, 4:1 as previously indicated) were incubated with a phycoerythrin (PE)-coupled anti-CD117 antibody (Biolegend, 313204, dil. Factor 1:50) in a final volume of 300 μl (incubated at 4° C., light protected for 10 min), followed by two washing rounds of centrifugation plus resuspension in 1 ml of fresh Column Buffer. The cell pellet was resuspended in 80 μl of Column Buffer. To that, 20 μl of Anti-PE MicroBeads UltraPure (Miltenyi, 130-105-639) were added, followed by a 15 min incubation (4° C., light protected). Cells were washed by centrifugation once and resuspended in Column Buffer to a final volume of 500 μl. All following steps were performed at room temperature.

### Flow-Through Collection

Cells were loaded on top of the equilibrated column (which remained bound to the MidiMACS Separator during the full length of this step). The column was washed twice by applying 2×1 ml of Column Buffer. The collected cell suspension from this step was kept in a separate tube and referred as non-bound cells hereafter.

### Eluate Collection

To eluate the cells that were magnetically bound to the column, the column was removed from the magnetic stand and placed over a fresh collection tube. 3 mL of Column Buffer was applied on top of the column, and the full content was collected on the same tube, referred as bound cells hereafter.

### Flow Cytometry Analysis

The contents of the two collected tubes (non-bound and bound cells) and the initial cell mixture were analyzed with a CytoFLEX B5-R3-V5 Flow Cytometer (Beckman Coulter). 6,000 cell events were recorded per sample, and the gating was defined by the presence of two very distinct population on the PE signal axis. Only single and live cells were included to this analysis. Data was analyzed with the software Kaluza C (Beckman Coulter) following manufacturer's instructions.

### Results and Conclusions

The aim of this experiment was to demonstrate the suitability and efficacy of a magnetic cell separation approach applied to a cell population mixture that contained keratinocytes and melanocytes, both being the most abundant cell types in human epidermis. This mixture resembles in composition the cell suspension that may be purified by the first and/or second purification material, as stated in the present specification.

The melanocyte-specific marker CD117 (C-Kit) is known to be absent in keratinocytes, which simplifies the cytometric readout to CD117 positive (High PE signal): melanocytes, and to CD117 negative (Low PE signal): keratinocytes. The correct initial composition of the cell mixture (keratinocytes:melanocytes, 4:1) was confirmed by flow cytometry before loading the cells into the column (FIG. 4A). Importantly, the non-bound cell fraction (“flow through”) was highly enriched in keratinocytes (99.84%, FIG. 4B) indicating that keratinocytes have none or very low affinity for the column, as expected (keratinocytes are not binding to anti-CD117 antibody). Contrary to this, the bound cell fraction was highly enriched in melanocytes (92.68%, FIG. 4C), depicting that melanocytes were retained by the separation matrix and were only eluted when the column was separated from the magnetic holder.

This method proved to be a reliable approach for the separation of melanocytes in a lab-made mixture of pure melanocytes and keratinocytes. In particular, adaptations to this protocol can be used to purify melanocytes present in human epidermal biopsies by using one or more of the hardware configuration(s) detailed in the present specification.

### LIST OF REFERENCE SIGNS

| | |
|---|---|
| Device | 1 |
| Main body | 2 |
| Layer | 3, 3a, 3b, 3c, 3d, 3e, 3f |
| Base | 4 |
| Vertical separator | 5 |
| Horizontal separator | 6 |
| Sub-compartment | 7a, 7b |
| Enzyme reservoir | 10 |
| Lid | 11 |
| Opening | 12 |
| First purification compartment | 20 |
| Second purification compartment | 30 |
| Syringe | 40 |
| Barrel | 41 |
| Piston | 42 |
| Syringe inlet | 43 |
| Syringe outlet | 44 |
| Barrel compartment | 45 |
| Injector | 50 |
| Support | 51 |
| Needle | 52 |
| Surgical tool | 60 |
| Rotating blade | 61 |
| Base | 62 |
| Pump inlet | 63 |
| Buffer reservoir | 70 |
| Waste reservoir | 80 |
| Conduit | 90, 90a, 90b, 90c, 90d, 90e |
| Valve | 91a, 91b, 91c, 91d |
| Buffer valve | 92 |
| Waste valve | 93 |
| Actuator | 94a, 94b, 94c, 94d, 94e |
| Magnet | 100 |
| Through-hole or gap | 101 |
| Mixing blade | 110 |
| Rod | 111 |
| Rotary actuator | 112 |
| Mixing chamber | 113 |
| Mixing device | 114 |
| Collecting chamber | 120 |
| Storage compartment | 130 |
| Enzyme solution | E |
| Inhibitor solution | I |
| First longitudinal axis | L1 |
| Second longitudinal axis | L2 |
| Magnetic particles | M |
| Tissue sample | T |
| First purification material | P1 |
| Second purification material | P2 |
| Waste solution | W |

1. A device (1) for preparing a cell suspension comprising
a. an enzyme reservoir (10) for containing an enzyme solution (E) capable of digesting components of a tissue sample (T) to obtain a cell suspension,
b. a first purification compartment (20) for receiving a first purification material (P1) capable of binding and/or retaining at least one component of the cell suspension, wherein the first purification compartment (20) is in fluid connection or can be brought into fluid connection with the enzyme reservoir (10),
c. a syringe (40) comprising a barrel (41) defining a barrel compartment (45) and a piston (42) which is movably arranged in the barrel compartment (45), wherein the syringe (40) comprises a syringe inlet (43) connected to the barrel compartment (45), and wherein the syringe (40) comprises a syringe outlet (44) for dispensing the cell suspension from the barrel compartment (45).
d. wherein the device (1) comprises a flow path from the enzyme reservoir (10) via the first purification compartment (20) to the syringe inlet (43).

2. The device (1) according to claim 1, characterized in that the device (1) comprises at least one valve (91) arranged in said flow path between the enzyme reservoir (10) and the syringe inlet (43), particularly wherein the at least one valve (91) is configured to allow flow in a first flow direction from the enzyme reservoir (10) through the first purification compartment (20) to the syringe inlet (43), and wherein the at least one valve (91) is configured to block flow in a second flow direction opposite the first flow direction.

3. The device (1) according to claim 1, characterized in that the device (1) comprises a second purification compartment (30) for receiving a second purification material (P2) capable of binding and/or retaining at least one component of the cell suspension, wherein the second purification compartment (30) is arranged in said flow path between the first purification compartment (20) and the syringe inlet (43).

4. The device (1) according to claim 1, characterized in that the device (1) comprises a buffer reservoir (70) for storing a buffer (B), wherein the buffer reservoir (70) is in fluid connection or can be brought into fluid connection with said flow path between the enzyme reservoir (10) and the syringe inlet (43) to mix said buffer (B) with the cell suspension, wherein particularly the device (1) comprises a buffer valve (92) connecting the buffer reservoir (70) to said flow path between the enzyme reservoir (10) and the syringe inlet (43).

5. The device (1) according to claim 1, characterized in that the device (1) comprises a waste reservoir (80) which is in fluid connection or can be brought into fluid connection with said flow path between the enzyme reservoir (10) and the syringe inlet (43) wherein particularly the device (1) comprises a waste valve (93) connecting the waste reservoir (80) to said flow path between the enzyme reservoir (10) and the syringe inlet (43).

6. The device (1) according to claim 1, characterized in that the device (1) comprises at least one magnet (100) arranged adjacent to said flow path between the enzyme reservoir (10) and the syringe inlet (43), wherein the at least one magnet (100) is configured to bind magnetic particles (M) comprised in the first purification material (P1) and/or the second purification material (P2).

7. The device (1) according to claim 1, characterized in that the device (1) comprises a mixing chamber (113) arranged in said flow path between the enzyme reservoir (10) and the syringe inlet (43), and a mixing device (114) configured to mix said cell suspension with said first purification material (P1) and/or said second purification material (P2).

8. The device (1) according to claim 1, characterized in that the device (1) comprises a surgical tool (60) for obtaining a tissue sample (T), wherein the surgical tool (60) is arranged adjacent to an opening (12) of the enzyme reservoir (10), particularly such that the tissue sample (T) obtained by the surgical tool (60) is automatically provided in the enzyme reservoir (10).

9. The device (1) according to claim 8, characterized in that the device (1) comprises a pump inlet (63) arranged in the enzyme reservoir (10), wherein the pump inlet (63) is configured to be connected to a vacuum pump to generate a vacuum in the enzyme reservoir (10), such that a portion of skin of a subject is sucked into the enzyme reservoir (10) by the vacuum if the portion of skin is placed on said opening (12) of the enzyme reservoir (10), and wherein the surgical tool (60) comprises at least one movable blade assembly (61) configured to cut off the portion of skin, thereby obtaining the tissue sample (T), wherein particularly the tissue sample (T) is provided in the enzyme reservoir (10) by the vacuum.

10. The device (1) according to claim 1, characterized in that the device (1) comprises an injector (50) comprising a plurality of hollow needles (52), wherein the injector (50) is connected to the syringe outlet (44), such that the cell suspension is dispensable from the barrel compartment (45) through the needles (52).

11. The device according to claim 10, characterized in that each of the needles has a length of 100 μm to 500 μm, particularly 100 μm to 200 μm.

12. A method for preparing a cell suspension comprising the steps of
providing a tissue sample (T), in the enzyme reservoir (10) of the device (1) according to claim 1,
providing an enzyme solution (E) in the enzyme reservoir (10),
incubating the tissue sample (T) in the enzyme solution (E) to digest components of the tissue sample (T), thereby generating a cell suspension,
providing the first purification material (P1) in the first purification compartment (20),
passing the cell suspension from the enzyme reservoir (10) through the first purification compartment (20) into the barrel compartment (45), such that at least one component of the cell suspension is bound or retained by the first purification material (P1), particularly by moving the piston (42) in the barrel compartment (45) and
dispensing the cell suspension from the syringe outlet (44), particularly by moving the piston (42) in the barrel compartment (45).

13. The method according to claim 12, wherein the first purification material (P1) is an affinity material capable of selectively binding at least one cell type, particularly keratinocytes or melanocytes, wherein more particularly the affinity material comprises antibodies which selectively bind to keratinocytes or melanocytes, wherein more particularly the first purification material (P1) comprises an inhibitor capable of inhibiting an enzymatic activity, particularly an enzymatic activity of an enzyme comprised in said enzyme solution (E).

14. The method according to claim 12, wherein the second purification material (P2) is provided in the second purification reservoir (20), and wherein after passing the cell suspension through the first purification compartment (20) and before dispensing the cell suspension from the syringe outlet (44), the cell suspension is passed through the second purification compartment (30) into the barrel compartment (45), such that at least one component of the cell suspension is bound or retained by the second purification material (P2), particularly by moving the piston (42) in the barrel compartment (45), wherein particularly the second purification material (P2) is a size exclusion material capable of separating components of the cell suspension according to their molecular weight and/or hydrodynamic radius.

15. The method according to claim 1, wherein the dispensed cell suspension comprises melanocytes, particularly wherein the dispensed cell suspension is free from keratinocytes.
